(19) United States
(12) Patent Application Publication
(10) Pub. No.: US 2013/0108679 A1
(43) Pub. Date: May 2, 2013

(54) PERSONAL CARE COMPOSITION

(71) Applicants: The Premiere Polish Company Limited, Cheltenham (GB); Aletheian Innovations Limited, Oxford (GB)

(72) Inventors: Andrew Butterfield, Cheltenham (GB); Paul Everett, Cheltenham (GB); Kenneth Jennings Barnes, Oxford (GB); Daniel Wingate Darg, Hereford (GB)

(73) Assignees: ALETHEIAN INNOVATIONS LIMITED, Oxford (GB); THE PREMIERE POLISH COMPANY LIMITED, Cheltenham (GB)

(21) Appl. No.: 13/660,331

(22) Filed: Oct. 25, 2012

Related U.S. Application Data
(60) Provisional application No. 61/619,974, filed on Apr. 4, 2012.

(30) Foreign Application Priority Data
Oct. 31, 2011 (GB) 1118786.1
Apr. 4, 2012 (GB) 1206043.0

(51) Int. Cl. A01N 25/08; A61K 36/886; B67D 7/06; A01N 33/12

(52) U.S. Cl. CPC A01N 25/08; A01N 33/12; A61K 36/886; B67D 7/06
USPC 424/414; 514/643; 424/744; 222/190

(57) ABSTRACT

According to the invention there is provided a foam or gel personal care composition having a disinfectant action including: 0.03-0.08% w/w of a biocide; a surfactant; a gelling agent; and water.

### CROSS-REFERENCE TO RELATED APPLICATIONS

This is a utility application which claims priority to Great Britain Application 1118786.1, filed Oct. 31, 2011, and Great Britain Application 1206043.0, filed Apr. 4, 2012, and the benefit of U.S. Provisional Application 61/619,974, filed Apr. 4, 2012, all of which are incorporated by reference herein in their entirety for all purposes.

### FIELD OF INVENTION

This invention relates to a personal care composition, a device for dispensing a personal care composition in foam or gel form, and a method of cleaning an external bodily area of a person.

### BACKGROUND OF THE INVENTION

Personal care products for cleaning an external bodily area of a person can take many forms. More recently, so called ‘wet-wipes’, which are moistened or impregnated cloths, have become popular, at least partly due to their ease of use. After use, it is common to dispose of wet wipes by flushing them down a toilet and into the sewerage system. However, wet wipes that are currently in the marketplace have a tendency to block sewerage pipework and septic tank systems because these products do not dissolve quickly enough.

The applicant has identified a need and desire for a convenient way in which to clean intimate body areas such as the external genital area and the region around the anus. Often, bodily cleaning of this type is performed in a bathroom or in a lavatory, such as prior to or after use of a toilet. Wet wipe products can be used for these purposes, but in these instances it is even more likely that the wet wipe would be disposed of by flushing down a toilet, with the undesirable consequences noted above. The applicant has also identified a need and desire to clean body areas of babies and young children, including intimate body areas, using a product which is both effective, pleasant and non-irritating. This is a particular problem with babies and young children.

It is well known to provide anti-bacterial gels for use in washing hands. However, in general products of this type would be unsuitable for use in cleaning intimate body areas such as the external genital area and the region around the anus. In part, this is because these body areas are sensitive to irritation. However, a suitable composition should be able to avoid irritation whilst providing sufficient cleaning and disinfectant action to satisfactorily clean these body areas.

International Publication WO2005/074947 discloses anti-viral pharmaceutical compositions which comprise two or more ionic components which give rise to an anti-viral action. However, these compositions are not intended for use as personal care products for cleaning purposes, and the compositions are not formulated for personal use. Instead, the compositions are intended as pharmaceutical compositions for medical purposes.

### SUMMARY OF THE INVENTION

The present invention, in at least some of its embodiments, addresses the above described problems, needs and desires. The present invention, in at least some of its embodiments, provides convenient personal care compositions and devices for dispensing personal care compositions which are well suited for use in cleaning an external bodily area of the person, with particular, but by no means exclusive, reference to the cleaning of intimate bodily areas such as the external genital area and around the anus.

### BRIEF DESCRIPTION OF THE DRAWINGS

FIG. 1 depicts a pump action foam dispenser useful to dispense the personal care composition described herein.

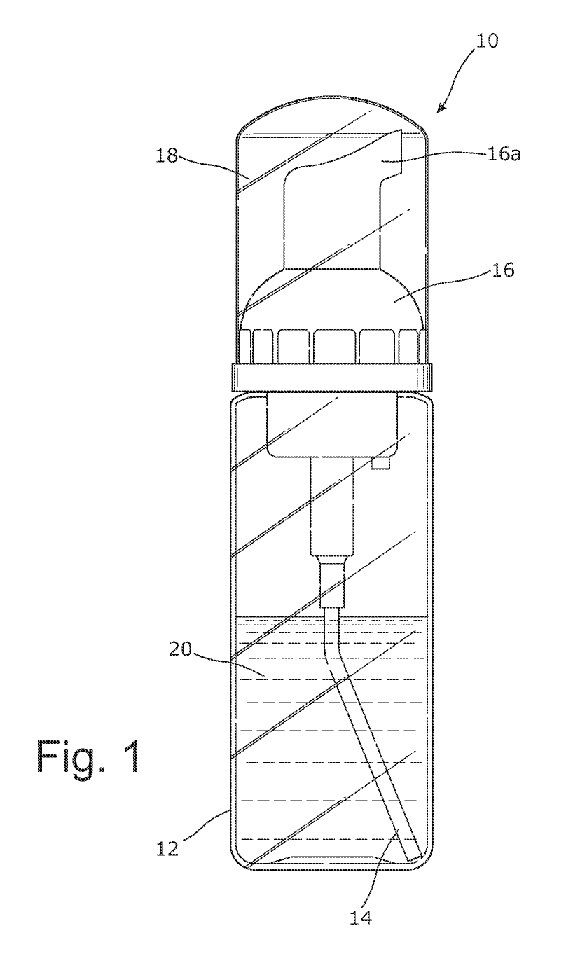

### DETAILED DESCRIPTION OF THE INVENTION

For the avoidance of doubt, it is understood that where reference is made herein to compositions or other features “including” one or more elements, the present specification also includes within its scope reference to compositions or other features “comprising”, “consisting essentially” and “consisting of said elements.

According to a broad aspect of the invention there is provided a foam or gel personal care composition having a disinfectant action including: a biocide; a surfactant; a gelling agent; and water.

The composition may include 0.01% w/w or greater, preferably 0.02% w/w or greater, more preferably 0.03% w/w or greater of the biocide.

The composition may include 5.0% w/w or less, preferably 0.2% w/w or less, more preferably 0.1% w/w or less of the biocide.

The composition may include 0.05% w/w or greater, optionally 0.1% w/w or greater, of the surfactant.

The composition may include 5.0% w/w or less of the surfactant.

The composition may include 0.1% w/w or greater, preferably 0.2% w/w or greater, more preferably 0.5% w/w or greater of the gelling agent.

The composition may include 10.0% w/w or less, preferably 2.0% w/w or less, more preferably 1.5% w/w or less of the gelling agent.

The composition may include 0.1% w/w or greater, generally 90% w/w or greater of water.

The composition may include 99.89% w/w or less of water.

With respect to any component in the composition, the invention also includes within its scope any combination of an upper limit and a lower limit described herein.

According to a first more specific aspect of the invention there is provided a foam or gel personal care composition having a disinfectant action including: 0.03-0.08% w/w of a biocide; a surfactant; a gelling agent; and water.

It has been found that compositions of this type are very well suited for personal care applications, particularly with regard to cleaning intimate body areas such as the external genital area and the region around the anus. In particular, the relatively low weight range of biocide utilised avoids irritation of sensitive areas of a user. Surprisingly, this relatively low amount of biocide also provides an effective disinfectant action.

The gel compositions of the invention may have a viscosity of 3,000-4,000 cps. This provides a pleasing feel when used.

Preferably, the composition includes 0.03-0.06% w/w of the biocide.

Gel personal care compositions of the invention may include the proviso that the gel composition does not include an ionic multivalent metal component and/or a cationic polymer.

In preferred embodiments, the biocide also acts as a surfactant, and there is no other surfactant present. This reduces irritancy, and provides a distinctive quality of foam. In particular, the foam can collapse quite rapidly to produce a pleasing feel on the skin. In other embodiments, the biocide acts as a surfactant, and there is an additional component which acts as a surfactant. In other embodiments still, the biocide does not act as a surfactant, and there is a separate component which acts as a surfactant.

Generally, the water acts as a solvent.

Preferably, the gelling agent is a cellulose-based gelling agent. The gelling agent may be a hydroxyethyl cellulose gelling agent, such a Natrosol (RTM) cellulose, or a methyl cellulose.

Advantageously, the biocide is a quaternary ammonium compound. A preferred example is benzalkonium chloride (BAC). It has been found that this biocide can perform a dual function by acting as a surfactant as well. This helps to provide a gentle cleansing action without irritation, even on sensitive skin. A further advantage is that satisfactory disinfectant properties can be obtained even with relatively low concentrations of the biocide in the composition (for example, at mixing ratios of 0.08% w/w or less). A further advantage still is that BAC can act as a preservative.

Other biocides, such amine-based antiseptics, might be contemplated. Examples of amine-based antiseptics are chlorhexidines, such as chlorhexidine gluconate.

The surfactant may be an amphoteric surfactant such as sodium cocoamphoacetate or a non-ionic surfactant such an alcoholic ethoxylate.

The composition may further include at least one of a humectant, a preservative, and a pH adjuster. Each of the humectant, preservative, and pH adjuster may be present in an amount between 0-5% w/w of the composition. The pH adjuster can be of any suitable form. In some embodiments, an acidic buffer may be used, whereas in the other embodiments an alkaline buffer may be used.

Any suitable preservative, such a isothiazalone or chlorhexidine gluconate may be utilised.

Any suitable humectant, such as those well known to the skilled reader, may be used in the composition. An example of a suitable humectant is glycerine.

Any suitable pH adjuster, such as would be commonly known to the skilled reader, may be utilised, such a citric acid or sodium hydroxide.

The composition may further include a moisturiser and/or an anti-irritant. The moisturiser and/or anti-irritant may be present in the composition in an amount 0-5% w/w, preferably 0.01-5.0% w/w, more preferably 0.1-0.5% w/w, most preferably about 0.2% w/w. The use of the moisturiser and/or anti-irritant can result in an enhanced cosmetic feel, especially when the composition is used as a foam. A particularly preferable silky feel can be imparted. The improved cosmetic feel is advantageous when the composition is intended for use on babies and/or young child.

An example of a moisturiser and/or anti-irritant is aloe vera. This imparts particularly desirable cosmetic feel, especially with regard to foam compositions. Compositions utilising aloe vera are excellent for use on babies and/or young children. Aloe vera may be present in the composition in an amount 0.01-5.0% w/w, preferably 0.1-0.5% w/w, more preferably about 0.2% w/w. The amounts are with respect to a 10× (ten times) concentrate of extracted aloe vera leaf juice. Alternatively, one or more of PEG-7 glyceryl cocoate, Chamomile, Avocado oil, sweet Almond oil might be used as a moisturiser. Other skin feel agents, emollients and occlusive materials might be used. Other examples of moisturisers and/or anti-irritants will readily suggest themselves to the skilled reader.

The composition may further include one or more further useful ingredients, such as a dye or a perfume. A perfume may be present in the composition in an amount 0-0.5% w/w. A dye may be present in the composition in an amount 0.1% w/w.

The dye can be any suitable water soluble and/or food grade dye. The perfume can be any suitable perfume having acceptable properties for use on human skin, such as a fragrant oil.

Preferably, the personal care composition is, in use, applied as a foam. This can be achieved by using a pre-cursor gel personal care composition of the type described above in combination with a foaming technique or foaming device of a type which is well known to the skilled reader.

Alternatively, the personal care composition may be applied, in use, as a gel. In these embodiments, the gel personal care composition may be supplied in a bottle or other container with a conventional outlet such a flip-top closure.

The foam or gel personal care composition may be dispensed from a portable container such as a bottle or a foaming dispenser, or from a permanently mounted dispenser such as a wall mounted dispenser. Touch-less dispensers may also be used to dispense the composition of the invention.

Personal care compositions of the invention can impart a pleasant feel or sensation and a velvety texture to the user after application. Composition can readily be provided which are skin safe, soap free, non-staining to clothes, and pH balanced. The compositions provide odour control by virtue of their disinfectant action. Compositions of the invention have been found to provide a log 3 kill (greater than 99.9% kill) of Staphylococcus Aureus and Esherichia Coli.

According to a second aspect of the invention there is provided a method of cleaning an external bodily area of a person, the method including the steps of: providing a personal care composition having a disinfectant action which includes 0.03-0.08% w/w of a biocide, a surfactant, and water;

and applying said composition to the external bodily area to provide a cleaning effect.

The composition can be used to freshen, cleanse and sanitise external bodily areas, including intimate external bodily areas. Advantageously, the composition does not require rinsing after application if used correctly. This makes the composition particularly suitable for use in areas when water is either a high value resource or is completely unavailable, such as camping, travel and festivals.

Advantageously, the external bodily area cleaned is the genital region or the anal region.

Preferably, the personal care composition is a foam or a gel personal care composition according to the first aspect of the invention.

Alternatively, the personal care composition may be a water thin composition including a biocide, a surfactant, and water. In these embodiments, it is not necessary for a gelling agent to be present. Other components described in relation to the first aspect of the invention may however be included in personal care compositions of this type. Water thin personal care compositions may be dispensed using a spray atomiser.

However, it is preferred that the personal care composition is applied either as a foam or as a gel.

Advantageously, the personal care composition is applied to a flexible substrate, wherein the cleaning effect is provided by wiping the external area with the flexible substrate.

Conveniently, the flexible substrate is a paper, preferably tissue paper. In particularly advantageous embodiments, the paper is dissolvable, and, after the step of applying said composition to the external bodily area, the composition and paper are disposed of by flushing in a toilet. In this way, an ecologically friendly way of cleaning is provided which does not block sewerage pipework and septic tank systems. This is in contrast to prior art wet-wipe products. A further advantage is that contact between the tissue or similar flexible substrate and the skin results in a gentle cleaning action. The area of contact can be wiped dry after use of the composition with a clean tissue.

Alternatively, the flexible substrate may be a cloth. The cloth may be a towel, flannel or other cloth which can be laundered for reuse after the cleaning has been performed.

Embodiments in which the composition is applied as a foam offer the advantage that the foam can be applied as discrete three-dimensional structure which tends to sit up on the flexible substrate and hence reduces absorption and disintegration of the flexible substrate. This effect is particularly pronounced when the flexible substrate is paper, particularly tissue paper.

The personal care composition may additionally be used to sanitise a hard surface, preferably a toilet seat.

The use of the personal care composition to clean a hard surface does not necessarily have to be performed in conjunction with the cleaning an external bodily area of a person. Accordingly, in a third aspect of the invention there is provided a method of cleaning a hard surface, preferably a toilet seat, the method including the steps of: providing a personal care composition having a disinfectant action which includes 0.03-0.08% w/w of a biocide, a surfactant, and water;

and applying said composition to the hard surface to provide a cleaning effect.

Preferably, the personal care composition is a foam or gel personal care composition according to the first aspect of the invention.

According to a fourth aspect of the invention there is provided a device for dispensing a foam personal care composition including: a chamber containing a personal care composition which includes 0.03-0.08% w/w of a biocide, a surfactant, a gelling agent, and water;

a foaming arrangement for mixing said composition with a gas supply to produce a foam; and

an outlet for the dispensing the foam.

The device can be of any form suitable for introducing a gas to a quantity of the personal care composition in order to produce a foam.

The device may be a pump-action device which includes a chamber which contains the personal care composition and a chamber which contains atmospheric air. The device may be of the type in which the pump-action causes the personal care composition and the air from the chambers through a nozzle to produce a foam. Other arrangements will readily suggest themselves to the skilled reader.

For the avoidance of doubt, the term “gas” is used herein is understood to include pure gases of a single chemical type, and mixtures of gases such as air.

The personal care composition in the chamber may be a gel composition according to the first aspect of the invention.

Whilst the invention has been described above, it extends to any inventive combination of the features set out above, or in the following description or claims.

Embodiments of personal care compositions and devices in accordance with the invention will now be described, by way of example only, with reference to the accompanying Figure, which is a semi-schematic diagram of a foamer device.

### EXAMPLES

An example of a gel personal care composition of the invention is described in the Table.

TABLE
Example of a personal care composition

| Component | % Wt/Wt | Function |
|---|---|---|
| 1. Water | 98.14 | solvent |
| 2. Acticide BAC50 | 0.08* | biocide/surfactant |
| (benzalkonium chloride) | | |
| 3. Aloe Vera 10x | 0.20 | moisturiser/cosmetic |
| 4. Glycerine | 0.75 | humectant, lubricant |
| 5. Perfume | 0.025 | perfume |
| Natures Touch 289571 | | |
| (Frag Oils) | | |
| 6. Natrosol 250 HHR/Tylose | 0.80 | gelling agent |
| H100,000 YP2 | | |
| 7. Citric acid | 0.005 | pH adjuster |

*BAC50 is a 50% active composition and therefore the active BAC biocide concentration is 0.04% Wt/Wt.

Manufacture

The components 1, 2 and 5 of the Table were sequentially added to a suitable vessel. Thereafter, component 6 was added, and the mixture stirred for 30-40 minutes or until component 6 has fully wetted out and thickened the mixture. Finally, component 7 was added to adjust the pH to the desired value.

Properties

Product specification

Product appearance: Clear, water white, viscous gel, fragrant

Specific Gravity: 1.010-1.020

Viscosity (Brookfield)

Spindle 2, Speed 3: 3,000-4,000 cps

Viscosity (Brookfield)

Spindle 2, Speed 6: 3,000-4,000 cps

pH (neat): 6.0-7.0

Refractive Solids (Bellingham Stanley RFM110): 1.5-2.5%

It is also possible to produce an unfragranced product by omitting component 5.

In tests, fragranced and unfragranced products of this type have been found to provide a log 3 kill (greater than 99.9% kill) of Staphylococcus Aureus ATTC 6538 and Esherichia Coli NCTC 10418.

The product can be applied as a gel for cleansing, or the gel product can be used as a precursor to the production of a foam which is used for cleaning purposes. The Figure shows a pump action foam dispenser, depicted generally at 10, comprising a body portion 12, a diptube 14, and a foam producing upper portion 16 including a dispensing nozzle 16a. The foam dispenser 10 further comprises a lid 18 which has a friction fit to the upper portion 16. The pump action foam dispenser is of a known, commercially available type and therefore further details of the device and its operation are not provided herein. Examples of suitable pump action foam dispensers include the F3 or M3 pump foamers sold by Rexam Airspray, NV (Ivoorstraat 9, 1812, RE Alkmaar, PO Box 1007, 1810 KA, Netherlands; www.rexam.com). Other suitable foam dispensers would readily suggest themselves to the skilled reader. The body portion 12 contains a suitable precursor solution 20, which is typically a gel composition such as the gel composition described in the Table. When the upper portion 16 is depressed by a user, the precursor solution 20 is drawn into the upper portion 16 via the diptube 14, and a foam personal care composition is dispensed from the nozzle 16a. The foam can have the advantages described above, including a pleasing and advantageous feel on the skin of a user.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A foam or gel personal care composition having a disinfectant action including:
0.03-0.08% w/w of a biocide;
a surfactant;
a gelling agent; and
water.

2. A foam or gel personal care composition according to claim 1 including 0.03-0.06 w/w of the biocide.

3. A foam or gel personal care composition according to claim 1 with the proviso that, if the composition is a gel composition, the gel composition does not include an ionic multivalent metal component and/or a cationic polymer.

4. A foam or gel personal care composition according to claim 1 in which the biocide also acts as the surfactant, and there is no other surfactant present.

5. A foam or gel personal care composition according to claim 1 including:
0.05-5.0% w/w of the surfactant; and/or 0.1-10.0% w/w of the gelling agent; and/or 0.1-99.89% w/w of water.

6. A foam or gel personal care composition according to claim 1 including:
0.2-2.0%, preferably 0.5-1.5%, w/w of the gelling agent; and/or greater than 90% w/w of water.

7. A foam or gel personal care composition according to claim 1 in which the gelling agent is a cellulose-based gelling agent.

8. A foam or gel personal care composition according to claim 1 in which the biocide is a quaternary ammonium compound.

9. A foam or gel personal care composition according to claim 8 in which the biocide is benzalkonium chloride.

10. A foam or gel personal care composition according to claim 1 further including a moisturiser and/or an anti-irritant.

11. A foam or gel personal care composition according to claim 10 in which the moisturiser and/or anti-irritant is present in an amount 0.01-5.0% w/w, preferably 0.1-0.5% w/w, most preferably about 0.2% w/w.

12. A foam or gel personal care composition according to claim 10 in which the moisturiser and/or anti-irritant is aloe vera.

13. A foam or gel personal care composition according to claim 11 in which the moisturiser is aloe vera and the amounts are with respect to a 10× concentrate of extracted aloe vera leaf juice.

14. A method of cleaning an external bodily area of a person, the method including the steps of:
providing a personal care composition having a disinfectant action which includes: 0.03-0.08% w/w of a biocide, a surfactant, a gelling agent, and water;
and applying said composition to the external bodily area to provide a cleaning effect.

15. A method of cleaning an external bodily area of a person according to claim 14 in which the external bodily area cleaned is the genital region or the anal region.

16. A method of cleaning an external bodily area of a person according to claim 14 in which the personal care composition is applied as a foam.

17. A method of cleaning an external bodily area of a person according to claim 14 in which the personal care composition is applied to a flexible substrate, wherein the cleaning effect is provided by wiping the external area with the flexible substrate.

18. A method of cleaning an external bodily area of a person according to claim 17 in which the flexible substrate is a paper, preferably tissue paper.

19. A method of cleaning an external bodily area of a person according to claim 18 in which the paper is dissolvable, and, after the step of applying said composition to the external bodily area, the composition and paper are disposed of by flushing in a toilet.

20. A device for dispensing a foam personal care composition including:
a chamber containing a personal care composition which includes 0.03-0.08% w/w of a biocide, a surfactant, a gelling agent, and water;
a foaming arrangement for mixing said composition with a gas to produce a foam; and
an outlet for dispensing the foam.
